(19) United States
(12) Patent Application Publication
(10) Pub. No.: US 2005/0224080 A1
(43) Pub. Date: Oct. 13, 2005

(54) A condom

(76) Inventor: Bo Lang, Shenzhen (CN)

Correspondence Address: BO LANG, 1401 HUATONG BUILDING, SUNGAG EAST ROAD, SHENZHEN, GD 518008, CN

(21) Appl. No.: 10/711,295

(22) Filed: Sep. 9, 2004

(30) Foreign Application Priority Data
Mar. 4, 2004 (CN) 2004100219848

(51) Int. Cl. A61F 6/02; A61F 6/04

(52) U.S. Cl. 128/844

(57) ABSTRACT

A condom, wherein the condom body is gradually enlarged in diameter along the hatch to the head direction, the condom loop is a thickened belt-like ring. The function of the loop is equivalent to the rim of the eye of a lamb that being adopted since ancient China. The loop can reduce the speed of the flow back of the blood in the vein of the penis but it does not holdback the pump in of the blood in the artery. Therefore it will increase the amount of the blood inside the sponge-like muscle of the penis. Working together with the condom body that gradually enlarged, it will bring the function similar to JELQ that being used on the adult ceremony in ancient Arabian world. Frequent adoption of this invention will enlarge penis.

### TECHNICAL FIELD

This invention relates to a condom, especially a condom that can enlarge penis.

### BACKGROUND ART

According to the prior art, some of the condoms have healthcare functions.

The problem common to all these ideas was that, none of them have penis enlargement functions.

There have been proposal for design which has a bigger condom head than the condom body, i.e., in China patent No. 00203229.5, titled An Interactive Condom, it proposes a condom with the head slightly bigger than the body part. But it's aimed to provide more comfort for the glans.

### SUMMARY OF THE INVENTION

It is therefore an object of this invention to provide a condom to control the blood circulation in & out of the penis. Further more, it enlarges the penis while the vagina controlling the to-and-fro motion of the penis working with the condom during sex.

The present invention in one embodiment provides a condom comprising condom body 1 and condom loop 2, wherein the condom body 1 is gradually enlarged in diameter along the hatch to the head direction. The condom loop 2 is a thickened belt-like ring.

According to this invention, the function of the loop is equivalent to the rim of the eye of a lamb that being adopted since ancient China. The loop can reduce the speed of the flow back of the blood in the vein of the penis but it does not holdback the pump in of the blood in the artery. Therefore it will increase the amount of the blood inside the sponge-like muscle of the penis. Just like doing gym often can enlarge muscles, the frequent congestion of the sponge-like muscle of the penis will lengthen and increase the fiber of the muscle of the penis. Working together with the condom body that gradually enlarged, it will bring the function similar to JELQ that being used on the adult ceremony in ancient Arabian world. This method increases the strength to expand the sponge-like muscle of the penis by extruding the penis forward from the root of the penis. During sex, the wall of the vagina is wrapping the penis. This to-and-fro motion is like JELQing. Therefore frequent adoption of this invention will enlarge penis.

### BRIEF DESCRIPTION OF THE DRAWINGS

FIG. 1 Shows the first embodiment of the present invention.

FIG. 2 Shows the second embodiment of the present invention.

FIG. 3 Shows the third embodiment of the present invention.

FIG. 4 Shows the forth embodiment of the present invention.

FIG. 5 Shows the fifth embodiment of the present invention.

FIG. 6 Shows the sixth embodiment of the present invention.

FIG. 7 Shows the seventh embodiment of the present invention.

### DETAILED DESCRIPTION OF THE DRAWINGS

This invention will now be described in further details with reference to the drawings.

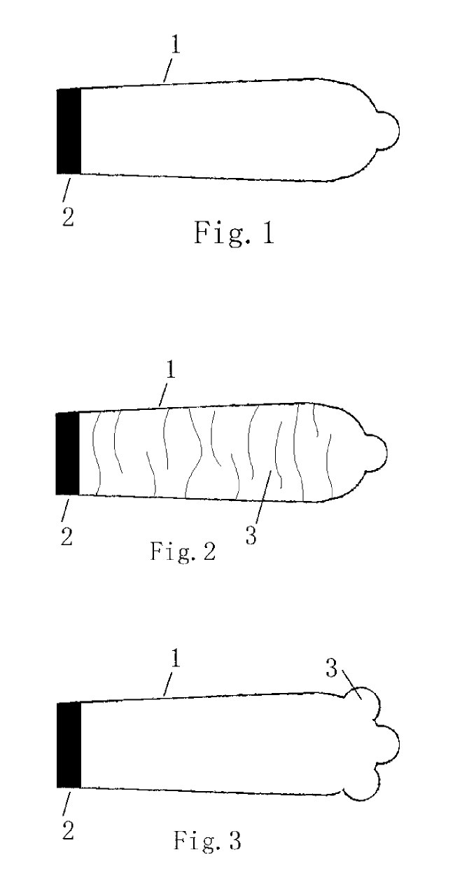

FIG. 1 to FIG. 5 show a condom, comprising condom body 1 and condom loop 2. As being shown in FIG. 1, the condom body here-of is gradually enlarged in diameter along the hatch to the head direction. The condom loop 2 here-of is a thickened belt-like ring. Certainly the condom loop can have an unchanged diameter or gradually enlarged in diameters. The alternative form of condom body can also have the same unchanged diameter as the condom loop. As being shown in FIG. 2, according to the principles of ergonomics and bionics, it's imitating the status when penis foreskin during sex to add continuing or break off corrugated heaves horizontally onto the outer wall of the condom body. Thus the penis in such a condom will give more realistic feelings to female. Modern medical science indicates that the points that will make women have orgasm are mainly the G point, P point and A point on the vagina vault in front of the womb opening. As being shown in FIG. 3, the present invention has one or multiple hollowed heaves 3 like sperm holders onto the head of the condom. The hollowed heaves 3 here-of are evenly laid on the outer circle. Hence it will increase the chances that G point P point and A point being touched. It will bring more chances that a female will get orgasm. Thus it will bring satisfying sexual life to couples, further more, a happy family. Modern medical science indicates that body fluid is the chief criminal for spreading sexual diseases. As being shown in FIG. 4, the present invention adds a skirt-like expanding cover in the shape of petal onto the condom hatch end. Therefore it will in a larger range decrease the area that sexual organs of the two persons being touched with each other to make it a much safer condom.

FIG. 5 is yet another embodiment of the present invention which is a combination of a condom body 1 that is gradually enlarged in diameter along the hatch to the head direction, a thickened belt-like loop 2 on the hatch of the condom, corrugated heaves 5 on the outer wall of the condom, hollowed heaves 3 on the head of the condom.

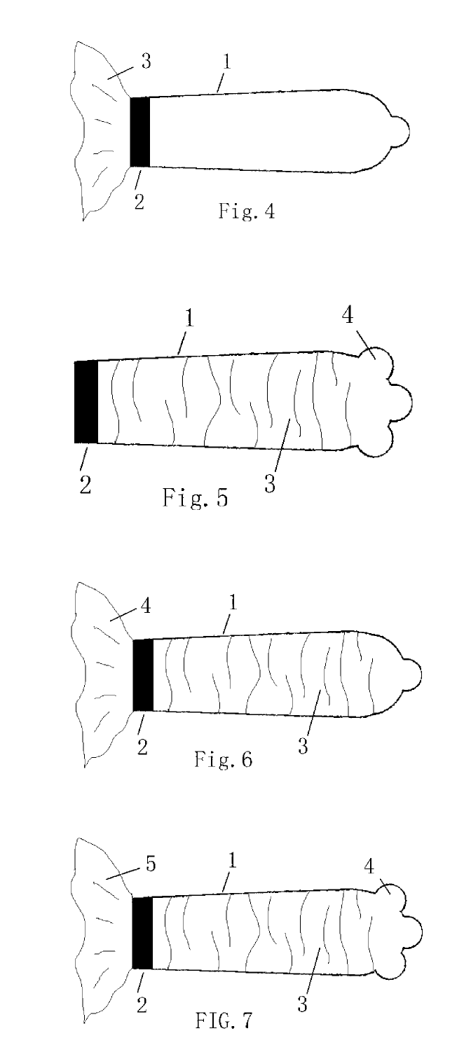

FIG. 6 is a further embodiment of the present invention which is a combination of a condom body 1 that is gradually enlarged in diameter along the hatch to the head direction, a thickened belt-like loop 2 on the hatch of the condom, corrugated heaves 5 on the outer wall of the condom, a skirt-like expanding cover 4 in the shape of petal onto the condom hatch end.

FIG. 7 shows the best mode for carrying out the present invention which will simultaneously adopt the condom body 1 that is gradually enlarged in diameter along the hatch to the head direction, a thickened belt-like loop 2 on the hatch of the condom, corrugated heaves 5 on the outer wall of the condom, hollowed heaves 3 on the head of the condom, a skirt-like expanding cover 4 in the shape of petal onto the condom hatch end.

Although the invention has been described by reference to particular illustrative embodiments thereof, many changes and modifications of the invention may become apparent to those skilled in the art without departing from the spirit and scope of the invention. It is therefore intended to include within this patent all such changes and modifications as may reasonably and properly be included within the scope of the present invention's contribution to the art.

1. A condom, comprising condom body 1 and condom loop 2, wherein the condom body is gradually enlarged in diameter along the hatch to the head direction. The condom loop 2 is a thickened belt-like ring.

2. The condom as defined in claim 1, wherein there is continuing or break off corrugated heaves horizontally on the outer wall of the condom body.

3. The condom as defined in claim 1 & claim 2, wherein there are one or multiple hollowed heaves at the condom head.

4. The condom as defined in claim 3, wherein the hollowed heaves are in the shape of a sperm holder.

5. The condom as defined in claim 3, wherein the hollowed heaves are evenly laid on the outer circle.

6. The condom as defined in claim 2 & claim 3, wherein there are expanding skirt-like cover in the shape of petal at the end of the condom hatch.
